(19) United States
(12) Patent Application Publication
(10) Pub. No.: US 2026/0021154 A1
(43) Pub. Date: Jan. 22, 2026

(54) HERBAL ANTI-INFAMMATORY COMPOSITION

(71) Applicant: CIRA HERBALS LLP, Rahanal, Bhiwandi, Thane (IN)

(72) Inventors: Gautam CHANDRAKUMAR SHAH, Mumbai (IN); Hiral MANITT SHAH, Mumbai (IN); Ritendre Singh MATHARU, Udaipur (IN)

(21) Appl. No.: 18/998,797

(22) PCT Filed: Jul. 29, 2023
(86) PCT No.: PCT/IN2023/050732

§ 371 Date: Jan. 27, 2025

(30) Foreign Application Priority Data
Jul. 29, 2022 (IN) 202221043574

(51) Int. Cl. A61K 36/535; A61K 9/08; A61K 9/20; A61K 9/48; A61K 36/28; A61K 36/59; A61K 36/71; A61P 29/00

(52) U.S. Cl. CPC A61K 36/535; A61K 9/08; A61K 9/20; A61K 9/48; A61K 36/28; A61K 36/59; A61K 36/71; A61P 29/00

(57) ABSTRACT

The present invention relates to a composition based on a mixture of herbal extracts of Nigella sativa (Ranunculaceae), Tinospora cordifolia (Menispermaceae), Petasites hybridus (Asteraceae), Perilla frutescens (Lamiaceae) for use as anti-inflammatory agent.

### FIELD OF THE INVENTION

The present invention relates to a composition based on a mixture of herbal extracts of Nigella sativa (Ranunculaceae), Tinospora cordifolia (Menispermaceae), Petasites hybridus (Asteraceae), Perilla frutescens (Lamiaceae) for use as anti-inflammatory agent.

### BACKGROUND OF THE INVENTION

Inflammation is a complex process, which is frequently associated with pain and involves occurrences such as the increase of vascular permeability, increase of protein denaturation, and membrane alteration. When cells in the body are damaged by microbes, physical agents, or chemical agents, the injury is in the form of stress. When tissue cells become injured, they release kinins, prostaglandins, and histamine. These events work collectively to cause increased vasodilation (widening of blood capillaries) and permeability of the capillaries. This leads to increased blood flow to the injured site. There is an increase in temperature at the injured site due to increasing blood flow. In response to this, the physiological proteins denature and further enhance the cascade of inflammatory mediators release (See N. Parrow et al. “Sequestration and Scavenging of Iron in Infection”, Infection and Immunity 81 no. 10 (2013):3503-3514). Inflammation is a bodily response to injury, infection or destruction characterized by heat, redness, pain, swelling, and disturbed physiological functions. It triggers the release of chemical mediators from injured tissue and migrating cells. The inflammatory mediators are histamine, 5-HT (serotonin), bradykinin, prostaglandins E2 (e.g., PGE2), interleukins, substance P, nitrous oxide, and the like (See Edvinsson, L., Haanes, K. A. & Warfvinge, K. Does inflammation have a role in migraine?; Nat Rev Neurol 15, 483-490 (2019)). In inflammatory disorders, there is excessive activation of phagocytes, production of O²⁻, OH radicals as well as non-free radicals species (H₂O₂), which can harm tissues severely either by powerful direct oxidation, or indirectly by lipid peroxidation resulting in membrane destruction (See S N Heendeniya, W D Ratnasooriya and R N Pathirana; In vitro investigation of anti-inflammatory activity and evaluation of phytochemical profile of Syzygium caryophyllatum, Journal of Pharmacognosy and Phytochemistry 2018; 7(1): 1759-1763). Tissue damage then provokes an inflammatory response by the production of mediators and chemotactic factors

There are references to use of the Nigella sativa in allergic rhinitis. Some of these are discussed here. WO0051580 is generally directed to the fields of medicine and pharmacology, and specifically directed to a pharmaceutical composition for the treatment of asthma/allergy, consisting essentially of Glycosphosphopeptical, or as an equivalent pure Nigella sativa seeds, which is active to stimulate T-helper lymphocytes type 1, therefore selectively switching-off the cosinophilic inflammation, also treating viral respiratory tract infections (flu & influenza), other viral infection, urinary tract infection, pelvic inflammatory diseases in particular neuroimmune appendicitis, cancer, Crohn's disease and facial palsy. CN111568955 discloses a Chinese medicinal composition for relieving allergic rhinitis characterized in that the active ingredients comprise black grass seeds, liquorice and fennel seeds; the mass ratio of the black grass seeds to the liquorice to the fennel seeds is 1-4:0.5-2:0.5 to 2. WO2020142054 discloses a nasal spray formulation containing Nigella sativa oil for use in the treatment of nasal dryness, nasal congestion and allergic rhinitis symptoms, stemming from dry air or influenza or common cold characterized in that, it comprises purified water, Nigella sativa oil, salt (NaCl), xylitol, eucalyptus oil, peppermint oil, emulgator and microbial preservative

JP2006070038 provides a natural product for use in a nutritional product, a dictary supplement, a food or a pharmaceutical composition used for treating allergies with a composition that comprises a Tinospora cordifolia extract. It discloses use in treating allergies (including but not limited to seasonal or perennial or sporadic allergic rhinitis).

Some prior art references are discussed here. DE19702168 discloses the use of extract of Petasites hybridus for the preparation of an agent for the treatment of allergic rhinitis. DE102004039011 discloses an extract comprising Petasites hybridus, which is free of pyrrolizidine alkaloid and furanoeremophilane. The extract is used for gastrointestinal, analgesic, antimigraine, antiasthmatic, anticonvulsant, antitussive, antiallergic, anti-inflammatory, anti-psoriatic, antiulcer, anti-arthritic, antirheumatic, immunosuppressive indications.

There are several references discussing the use of Perilla frutescens in allergic rhinitis. Some of these are discussed here. US2019255135 relates to a composition for treatment of an inflammatory disease comprising a composite plant extract including a Perilla frutescens extract and an Atractylodes macrocephala Koidzumi extract. KR20200052628 discloses a pharmaceutical composition for the prevention or treatment of inflammatory diseases or allergic diseases containing supercritical defatted perilla powder cthanol extract as an active ingredient. JPH09291299 claims an oil and fat containing ω3 polyunsaturated fatty acids, an oil and fat containing ω06 polyunsaturated fatty acids, and a perilla leaf extract for use as an antiallergic oil and fat composition. US10238707 claims a composition comprising at least one slow-release component comprising quercetin; and at least one fast release component comprising an extract from Perilla frutescens or a constituent thereof selected from luteolin, rosmarinic acid, apigenin, catechin, acid, caffeic acid or mixture thereof. KR102048977 discloses an anti-allergic composition using an extract of Ulmus davidiana bark and an extract of Perilla frutescens, which can be used in treating atopic dermatitis, asthma, allergic rhinitis, allergic conjunctivitis, allergic dermatitis, allergic contact dermatitis, or allergic keratitis. CN110613775 discloses a traditional Chinese medicine formula for treating rhinopathy prepared from the raw materials including Perilla frutescens, along with several other herbal extracts.

While the individual herbs have been reported in the art for treating allergic rhinitis, and/or have been disclosed to be useful in combination with other herbs, there is nothing in the art that suggests a composition containing the specific combination of Nigella sativa (Ranunculaceae), Tinospora cordifolia (Menispermaceae), Petasites hybridus (Asteraceae), and Perilla frutescens (Lamiaceae) for use in treating inflammation.

Inflammation leads to commonly seen conditions such as joint pain or arthritis, allergy, asthma, allergic rhinitis, autoimmune diseases, coceliac disease, glomerulonephritis, hepatitis, inflammatory bowel disease, pre-perfusion injury, transplant rejection and some others. Most common is allergy and allergic rhinitis, as well as pain of the joints. Typical standard therapy for such inflammation are agents such as ibuprofen, paracetamol, cetirizine, cinnarizine, fexofenadine and the like. The present invention now provides a polyherbal mixture as an effective agent for treating inflammation, such as allergic rhinitis and joint pain.

### OBJECTIVE OF THE INVENTION

It is an object of the invention to provide a composition based on herbal extracts for use as an anti-inflammatory agent.

A further object of the invention is to provide an herbal anti-inflammatory composition which is safe and does not suffer from the limitations of known compositions based on synthetic compounds, which cause several side effects, including drowsiness, acid reflux and the like

Another object of the invention is to provide an herbal anti-inflammatory composition which is a polyherbal extract that is effective at a concentration lower than that required for each individual component of the polyherbal extract to provide anti-inflammatory effect.

A still further object of the invention is to provide an herbal anti-inflammatory composition which is effective in the treatment and management of allergies, such as allergic rhinitis.

### SUMMARY OF THE INVENTION

The present invention provides an anti-inflammatory composition based on herbal extracts wherein the composition comprising a mixture of—
(i) water extract of Nigella sativa,
(ii) water extract of Tinospora cordifolia,
(iii) water extract of Petasites hybridus,
(iv) water ext ract of Perilla frutescens

In one embodiment, the anti-inflammatory composition comprises about 20% to about 35% w/w of water extract of Nigella sativa, about 40% to about 60% w/w of water extract of Tinospora cordifolia, about 5% to about 15% w/w of water extract of Petasites hybridus, and about 5% to about 20% w/w of water extract of Perilla frutescens.

In yet another other embodiment, the anti-inflammatory composition comprises about 0.12 μg to about 80 μg of Nigella sativa, about 0.5 μg to about 10 μg of Tinospora cordifolia, about 0.12 μg to about 20 μg of Petasites hybridus, about 0.25 μg to about 10 μg of Perilla frutescens.

In yet another embodiment, the anti-inflammatory composition comprises a mixture of—
(i) about 20% to about 35% w/w of extract of Nigella sativa,
(ii) about 40% to about 60% w/w of extract of Tinospora cordifolia,
(iii) about 5% to about 15% w/w of extract of Petasites hybridus,
(iv) about 5% to about 20% w/w of extract of Perilla frutescens.

In a further embodiment, the polyherbal extract comprising a mixture of Nigella sativa, Tinospora cordifolia, Petasites hybridus, and Perilla frutescens is formulated into a suitable dosage form for oral administration in the treatment and/or prophylaxis of an inflammatory condition.

### BRIEF DESCRIPTION OF DRAWINGS

FIG. 1 describes the inhibition of protein denaturation by Nigella sativa (Ranunculaceae; D1)

FIG. 2 describes the inhibition of protein denaturation by Tinospora cordifolia (Menispermaceae; D2)

FIG. 3 describes the inhibition of protein denaturation by Petasites hybridus (Asteraceae; D3)

FIG. 4 describes the inhibition of protein denaturation by Perilla frutescens (lamiaceae; D4)

FIG. 5 describes the inhibition of protein denaturation by Ibuprofen

FIG. 6 describes the inhibition of protein denaturation by Cetrizine

FIG. 7 describes the inhibition of protein denaturation by Fexofenadine

FIG. 8 describes the inhibition of protein denaturation by polyherbal aqueous extract

In the graph, X axis represents concentration in ug and Y axis represents percent inhibition of protein coagulation and n=3.

IC50 (i.e., x in the equation) value for each extract and standard was thus determined from equation of the graph (y=mx+c, wherein y is 50% inhibition of protein coagulation, m is slope of the line, x is IC50 and c is y-intercept of the line.

### DETAILED DESCRIPTION OF THE INVENTION

The term “comprising”, which is synonymous with “including”, “containing”, or “characterized by” herein defined as being inclusive or open-ended and does not exclude additional, unrecited elements or method steps, unless the context clearly requires otherwise.

The term “about” means within 20%, 15%, 10%, 9%, 8%, 7%, 6%, 5%, 4%, 3%, 2%, 1%, 0.5%, 0.1%, or 0.05% of a given value or range.

Allergic rhinitis (AR) is the most common of all allergic disease affecting approximately 400 million people worldwide and is associated with a reduced quality of life of the patients, lower work productivity and school learning performance as well as increasing medical costs. Allergic rhinitis is an IgE-mediated inflammatory disease of the nasal mucous membranes due to the interaction of allergen characterized by an inflammatory infiltrate made up of Eosinophils, T cells, mast cells and Basophils, which release several mediators, chemokines and cytokines (among these, histamine and cysteinyl-leukotrienes are the major vasoactive mediators), regulation of the local and systemic IgE synthesis, and communication with the immune system and the bone marrow.

The present invention provides an anti-inflammatory composition based on herbal extracts wherein the composition comprises a mixture of—
(i) extract of Nigella sativa,
(ii) extract of Tinospora cordifolia,
(iii) extract of Petasites hybridus,
(iv) extract of Perilla frutescens
   Nigella sativa (N. sativa)

It is commonly known as black caraway, black cumin, black seed, Roman coriander, fennel flower, nigella or kalonji, is an annual flowering plant in the family Ranunculaceae, native to eastern Europe (Bulgaria and Romania) and Western Asia (Cyprus, Turkey, Iran and Iraq). It is considered to be densely packed with nutrients and is traditionally known to provide numerous health benefits in treating respiratory disorders, cardiovascular disorders, mental debility, rheumatism, inflammation, back pain, anorexia, amenorrhea, paralysis, etc. It is regarded as a wonder drug by most practitioners of traditional medicine. The seeds of N. sativa contain a substantial quantity of proteins which contain amino acids such as glutamate aspartate, methionine, arginine and cysteine, fibre, fats, and carbohydrates as well as several vitamins and minerals. N. sativa seed fixed oil known to be used in inflammatory disorders like sinusitis, otitis, bronchitis or asthma. The anti-inflammatory effect of N. sativa is probably produced by reduction of cicosanoids synthesis (thromboxane A2, prostaglandins and leukotrienes), due to inhibition of COX and lipoxygenasc.

Tinospora cordifolia (T. cordifolia),

It is commonly known as gurjo, heart-leaved moonsced, guduchi or giloy, is a herbaceous vine of the family Menispermaceac indigenous to tropical regions of the Indian subcontinent. It has been in use for centuries in traditional medicine to treat various disorders. All the parts of the plant are immensely useful due to the presence of different compounds of pharmaceutical importance belonging to various groups as alkaloids, diterpenoid lactones, glycosides, steroids, sesquiterpenoid, and phenolics. These compounds possess pharmacological properties, which make it anti-diabetic, antipyretic, anti-inflammatory, anti-oxidant, hepato-protective, and immuno-modulatory. Root extract of T. cordifolia known to have a strong anti-inflammatory effect. The anti-inflammatory effect of T. cordifolia was mediated via reduction of the proinflammatory cytokines such as: IL-1β, TNF-α, IL-6, and IL-17; the frequency of IL-17-producing T cells; and the production of chemokines such as RANTES (Regulated upon Activation, Normal T Cell Expressed and Presumably Secreted).

Petasites hybridus (P. hybridus)

It is commonly known as the butterbur, is an herbaceous perennial flowering plant in the family Asteraceae, native to Europe and northern Asia. Although used over centuries in traditional medicine to treat various disorders, there are no approved medical uses but it is sold as a dietary supplement. It was traditionally used for the treatment of malignant and cancerous ulcers, migraines, gastrointestinal, respiratory and genitourinary disorders. Butterbur generally consists of various constituents which include sesquiterpenes belonging to three major classes-bakkenolides, cremophilane and furanocremophilanes. Pyrolizidine alkaloids and phenolic compounds are also present. Butterbur root extract known to treat migraines in adults and also shows potential in the treatment of pediatric migraines. Other uses include the treatment of asthma, allergic rhinitis etc. It is also used as a spasmolytic, neuroprotective, cardio-protective, anti-inflammatory, antioxidant etc. and may be used as a functional food. The anti-inflammatory activity of P. hybridus root extract is attributed to its sesquiterpene ester components, such as petasin and isopetasin. Butterbur decreases the production of the inflammatory mediators prostaglandin E2 (PGE2), leukotriene B4 (LTB4) and cysteinyl-leukotrienes (LTs), in animal and human cellular system, as well as purified enzyme preparations.

Perilla frutescens (P. frutescens)

It is commonly known as deulkkae, perilla or Korean perilla, is a species of Perilla in the mint family Lamiaceae. It is an annual plant native to Southeast Asia and Indian highlands, and is traditionally grown in the Korean peninsula, southern China, Japan and India as a crop. Constituents present in Perilla include Rosmarinic acid, Luteolin, Chrysocriol, Quercetin, Catechin, Caffeic acid, Ferulic acid and several other compounds. P. frutescens seeds contain oil, protein, sterols such as phytosterol and tocopherol, squalene, and polyunsaturated fatty acids. Its leaves contain abundant quantities of carotenoids. The oil obtained from the seeds demonstrates anti-oxidant, anti-inflammatory, antimicrobial, antifungal and anti-cancer activity. Other pharmacological actions include anti-spasmodic, anti-depressant, neuroprotective, insecticidal, hepatoprotective and anti-allergic activity. Each of these activities can be attributed to the numerous phytoconstituents present. Several anti-inflammatory components of perilla leaf have also been identified, including luteolin and tormentic acid known to suppress the overproduction of tumor necrosis factor alpha, a cytokine important in immunologic and inflammatory reactions.

While the individual herbs have been reported in the art for treating allergic rhinitis and/or inflammation, there is nothing in the art that suggests a composition containing the specific combination of Nigella sativa (Ranunculaceae), Tinospora cordifolia (Menispermaceae), Petasites hybridus (Asteraceae), and Perilla frutescens (Lamiaceae) for use in treating inflammation.

A water extract of the four individual herbs, i.e., Nigella sativa (Ranunculaceae), Tinospora cordifolia (Menispermaceae), Petasites hybridus (Asteraceae), and Perilla frutescens (Lamiaceae), was obtained by methods conventional in the art, and which are commercially viable. The individual extracts were collected and stored under conditions such that they remain physically and chemically stable. By stable is meant that there is no microbial growth and there is no precipitation seen upon storage.

A polyherbal extract was obtained by mixing (i) about 20% to about 35% w/w of water extract of Nigella sativa, (ii) about 40% to about 60% w/w of water extract of Tinospora cordifolia, (iii) about 5% to about 15% w/w of water extract of Petasites hybridus, and (iv) about 5% to about 20% w/w of water extract of Perilla frutescens. This polyherbal extract was tested for anti-inflammatory effects, as described in the examples below. The terms “polyherbal extract” and “polyherbal mixture” may be used interchangeably.

The reactive oxygen species are also known to activate matrix metalloproteinase damage seen in various arthritic tissues. Inflammation is a protective response to tissue injury and it involves a complex array of enzyme activation, mediator release, cell migration, fluid extravasations, tissue breakdown, and repair, The rationale behind implementing this assay is that denaturation of albumin proteins leads to the formation of antigens which initiates type III hypersensitive reaction leading to inflammation. And therefore, inhibition of its denaturation process by an agent indicates its anti-inflammatory properties; the higher the degree of inhibition of protein denaturation greater would be its anti-inflammation potential (See Jayashree V, Bagyalakshmi S, Manjula Devi K, Richard Daniel; In vitro anti-inflammatory activity of 4-benzylpiperidine, Asian J Pharm Clin Res, Vol 9, Suppl. 2, 2016, 108-110).

Protein Denaturation is a process in which proteins lose their tertiary structure and secondary structure by application of external stress or compound, such as strong acid or base, a concentrated inorganic salt, an organic solvent, or heat. Most biological proteins lose their biological function when denatured. Denaturation of proteins is a well-documented cause of inflammation. This principle was used to conduct in vitro testing of the individual components and the polyherbal mixture, to detect the IC50 valuc, i.e., amount required to cause 50% inhibition of protein denaturation.

Herbs were chosen according to quality inspection (differentiation and authentication of the raw materials, heavy metal analysis, pesticide residue check and active ingredient content analysis). Seeds of Nigella sativa, stem bark of Tinospora cordifolia, arial parts of Petasites hybridus and seeds of Perilla frutescens were chosen for extraction of their components. Sliced herbal material, i.e. stem bark and arial parts, where used, were dried in oven at a temperature not exceeding 50° C. Following size reduction in a grinder mill, these were sieved through 80 mesh. Required amount of powdered drug was macerated with sufficient quantity of suitable solvent(s) after maceration. The seeds were crushed, powdered and subjected to maceration, followed by suspension in a solvent and subjecting it to ultrasonification. The resultant extracts were filtered, and the filtrate concentrated to dryness. While dry powders were obtained for Nigella sativa, Tinospora cordifolia and Petasites hybridus, a semi-solid extract was obtained for Perilla frutescens, as it contained a significant quantity of oils.

The solvents that are suitable for obtaining the extract of the herbs mentioned above, include but are not limited to, water, ethanol, propanol, isopropanol, dimethyl formamide, dimethyl sulfoxide, methylene dichloride, acetone, hexane, diethyl ether and other solvents conventionally used in the extraction of herbal products. These solvents can be used alone or in admixture, in various quantities, as may be required. A skilled person is adept with the knowledge of assessing the suitable solvent needed for the extraction, and can use the same (See Abubakar et al, J Pharm Bioallied Sci. 2020 January-March; 12(1): 1-10). In preferred embodiments of the present invention, the solvent used is selected from water, alcohol or mixtures thereof. In highly preferred embodiments, water is used as the solvent for extraction of Nigella sativa, Tinospora cordifolia, Petasites hybridus and Perilla frutescens. The individual extracts thus obtained are then mixed in defined amounts to obtain the polyherbal extract of the present invention. In preferred embodiments, 0.12 μg to about 80 μg of Nigella sativa, about 0.5 μg to about 10 μg of Tinospora cordifolia, about 0.12 μg to about 20 μg of Petasites hybridus, about 0.25 μg to about 10 μg of Perilla frutescens are mixed to obtain the polyherbal extract.

Macroscopic identity of herbal materials is based on shape, size, colour, surface characteristics, texture, fracture characteristics and appearance of the cut surface. However, since these characteristics are judged subjectively and substitutes or adulterants may closely resemble the genuine material, it is often necessary to substantiate the findings by microscopy and/or physicochemical analysis. The herbal extract was further evaluated for foreign organic matter, total ash content, loss on drying, as well as extractable value by conventional methods known in the art.

Each individual extract was standardised so as to ensure reproducibility of the final product to be used in the polyherbal extract of the invention. Biomarkers were identified for the polyherbal extract, and the same were analysed using methods standard in the art. For the polyherbal extract of the present invention, four biomarkers were selected for standardisation, viz. Rosamarinic acid, Berberinc, Petasin and Thymoquinone. The polyherbal extract of the invention thus has standardised quantities of these biomarkers, which are bioactive components of the extract. Standardisation ensures that the polyherbal extract contains the same amount of the active everytime it is prepared. This is important in herbal preparations because the quantity of the actives can vary based on the content in the plant, stem bark, arial parts or seeds, depending on the quantity used, as well as based on environmental factors that can impact the natural content of the actives in these herbs. If the process of standardisation is not conducted, then it is very likely that there will be batch-to-batch variations, i.e. the content of the actives may vary from one batch to another, thereby impacting the anti-inflammatory activity of the polyherbal extract. Thus, the present invention provides for the first time a standardised extract of the four herbs for anti-inflammatory use.

The present invention provides a polyherbal extract containing Nigella sativa, Tinospora cordifolia and Petasites hybridus and Perilla frutescens, wherein the anti-inflammatory effect of the combination is significantly higher than that obtained with cach individual extract. As can be seen in the examples provided below, the polyherbal extract provides synergy in the treatment of inflammation, i.e. the amount of the polyherbal extract required to provide an anti-inflammatory effect (measured in terms of IC50, as described above) will be much lower than the amount of each individual extract required to provide the same effect. Further, the polyherbal extract of the present invention also provides better anti-inflammatory effect than known standard drugs such as ibuprofen, fexofenadine and cetirizine. The polyherbal extract is therefore expected to provide a better effect than the individual herbs, as well as the standard drugs.

The polyherbal extract of the invention may be further formulated with suitable pharmaceutically acceptable excipients to obtain dosage forms such as capsules, tablets, granules, powders filled in sachet, oral liquid solutions, oral liquid suspensions, dry suspensions for reconstitution, and the like. Typically, excipients such as binders, diluents, disintegrants, lubricants, flow-enhancing agents, solubility enhancing agents, permeability enhancing agents, sweeteners, flavorants, coating agents and the like may be used. The amount of these excipients can be appropriately set depending on the kind of the excipient used, and as known in the pharmaceutical art.

In a preferred embodiment, the composition is formulated as tablets.

In another preferred embodiment, the composition is formulated as capsules. The capsule may contain powders, granules, pellets or mini-tablets.

The composition may contain binders that may be selected from, but are not limited to, starch, pregelatinized starch, sodium alginate, gelatin, polyvinyl pyrrolidone (PVP), methylcellulose, hydroxy propyl methyl cellulose (HPMC), polymethacrylates, sodium carboxy methyl cellulose, polyethylene glycol (PEG) and methylcellulose, or mixtures thereof. The binder is present in an amount of about 0.1% to about 5% by weight of the composition.

The composition may also contain fillers selected from disaccharides such as lactose and sucrose; polysaccharides and their derivatives such as starches, cellulose, or modified cellulose such as microcrystalline cellulose (MCC) and cellulose ethers such as hydroxypropyl cellulose (HPC); sugar derivatives such as sorbitol, xylitol, and mannitol, etc. The fillers may be present in an amount from about 5% to about 95% by weight of the composition.

The composition may include disintegrants selected from the group comprising croscarmellose sodium, starch, sodium starch glycolate, crospovidone, carboxymethyl cellulose calcium, carboxymethylcellulose sodium, magnesium aluminium silicate or mixtures thereof, and more preferably croscarmellose sodium and/or starch. The disintegrant may be present in an amount of about 0.1% to about 20% by weight of the composition.

The composition may also include flow-enhancing agents in an amount from about 0.1% to about 5% by weight of the composition. The flow-enhancing agents may be for example colloidal anhydrous silica, silicic acid, tale or mixtures thereof.

The composition may also contain a lubricant in an amount from about 0.1% to about 5% by weight of the composition. Examples of lubricants for use in accordance with the present invention include but are not limited to magnesium stearate, calcium silicate, talc, fumed silicon dioxide, calcium stearate, stearic acid, sodium stearyl fumarate. Preferably, the lubricant, if present, is magnesium stearate or stearic acid, more preferably magnesium stearate.

The composition may optionally contain a solubility enhancing agent such as medium chain fatty acid or a salt, ester, ether or derivative of a medium chain fatty acid having a carbon chain length of about 4 to about 20 carbon atoms. An absorption enhancing agent, if present, may be selected from one or more of sodium caprate, sodium dodecanoate, sodium palmitate, chitosan and derivatives thereof, fatty acids, surfactants, liposomes, triglycerides, polyethers, bile salts, triglycerides, and mixtures thereof.

Sweeteners or sweetening agents that may be used in the compositions of the present invention include any compound that provides a sweet taste. This includes but is not limited to nutritive sweetening agents, non-nutritive sweetening agents and mixtures thereof. The nutritive sweetening agents may be selected from, but are not limited to, dextrose, fructose, sucrose, brown rice syrup, date sugar, honey, maple syrup, coconut sugar, erythritol, maltitol, mannitol, sorbitol, xylitol, isomalt, lactitol, and mixtures thereof. The non-nutritive sweetening agents may be selected from, but are not limited to, acesulfame, aspartame, neotame, saccharin, sodium saccharin, sucralose, acesulfame potassium, stevioside and mixtures thereof.

A flavoring agent or flavorant may be added to enhance the taste or aroma of the composition of the present invention. Non-limiting examples of suitable natural flavors, some of which can readily be simulated with synthetic agents or combinations thereof, include almond, anise, apple, apricot, bergamot, blackberry, blackcurrant, blueberry, cacao, caramel, cherry, cinnamon, clove, coffee, coriander, cranberry, cumin, dill, eucalyptus, fennel, fig, ginger, mango, grape, grapefruit, guava, hop, lemon, licorice, lime, malt, mandarin, molasses, nutmeg, mixed berry, orange, peach, pear, peppermint, pineapple, raspberry, rose, spearmint, strawberry, tangerine, tea, vanilla, winter green, and the like, as well as combinations thereof.

The composition of present invention may have one or more coatings such as film coating or sugar coating. Coating agents which are useful in the coating process, include, but are not limited to, polysaccharides, alkyl celluloses such as methyl or ethyl cellulose, hydroxyalkylcelluloses (e.g. hydroxypropylcellulose or hydroxypropyl-methylcelluloses); polyvinylpyrrolidone, acacia, corn, sucrose, gelatin, shellac, polyvinyl alcohol (PVA), copolymers of vinylpyrrolidone and vinyl acetate (e.g. marketed under the brand name of plasdone).

The composition of the present invention was assessed using milk-induced cosinophilia and leukocytosis animal model to evaluate the anti-inflammatory activity.

Injection of milk into mice induces allergy response which in turn causes leucocytosis. Leukocytosis is the condition of having a high number of leukocytes in the blood. Leukocytes are white blood cells. Leukocytosis may be a temporary response to an allergy, infection or an injury as the body is healing naturally, or it can be a sign of disease.

Cell viability is defined as the number of healthy cells in a sample. The measurement of cell viability plays an important role for all forms of cell culture. Cell viability assays are essentially used for screening the response of the cells against a drug or a chemical agent. In particular, pharmaceutical industry widely uses viability assays to evaluate the influence of developed agents on the cells.

There are several types of assays that can be used to determine the number of viable cells. These assays are based on various functions of cells including enzyme activity, cell membrane permeability, cell adherence, adenosine triphosphate (ATP) production, co-enzyme production, and nucleotide uptake activity. Although there are different classifications, cell viability assays may be broadly categorized as (a) dye exclusion assays, (b) colorimetric assays, (c) fluorometric assays, (d) luminometric assays, and (c) flow cytometric assays. Dye exclusion assays are the simplest methods that are based on utilization of different dyes such as trypan blue, cosin, congo red, and erythrosine B, which are excluded by the living cells, but not by dead cells.

Trypan blue stain assay has been developed to measure viable cell count and is used as a confirmatory test for measuring changes in viable cell number caused by a drug or toxin. Trypan blue stain, a large negatively charged molecule, is one of the simplest assays that are used to determine the number of viable cells in a cell suspension. The principle of this assay is that living cells have intact cell membranes that exclude the trypan blue stain, whereas dead cells do not. Cell suspension is mixed with the trypan blue stain and examined visually under light microscopy to determine whether cells include or exclude the stain. A viable cell will have a clear cytoplasm, whereas a nonviable cell will have a blue cytoplasm.

The following examples describe the nature of the invention and are given only for the purpose of illustrating the present invention in more detail and are not limited and relate to solutions, which have been particularly effective on bench scale.

### Example 1

Nigella sativa seeds, Tinospora cordifolia stem bark, Petasites hybridus arial parts and Perilla frutescens seeds were extracted with water. These extracts are described in Table 1 below:

TABLE 1

| Name of Extract | Colour | pH | Solubility | Appearance |
|---|---|---|---|---|
| Nigella sativa | Blackish | 6 | Soluble in | Free-flowing |
| (seeds) | brown | | water | powder |
| Tinospora | Dark brown | 7 | Soluble in | Free-flowing |
| cordifolia (root) | | | water | powder |
| Petasites hybridus | Brown | 6 | Soluble in | Free-flowing |
| (root) | | | water | powder |
| Perilla frutescens | Greyish brown | 6 | Soluble in 0.1% | semisolid |
| (seeds) | | | Tween 80 | |

A polyherbal extract was obtained by mixing the 4 extracts in the amounts mentioned in Table 2 below:

TABLE 2

| Name of Extract | Quantity (% w/w) |
|---|---|
| Nigella sativa | 28.59 |
| Tinospora cordifolia | 51.36 |
| Petasites hybridus | 8.56 |
| Perilla frutescens | 11.47 |

### Example 2

### 1. Formulation of Polyherbal Tablets

### A. Preparation of Blend for Tablet

The extracts of all four herbs were mixed and the blend was passed through #40 mesh sieve to ensure uniformity of the particles. There were no excipients added.

### B. Pre-Compression Parameters of the Blended Mixture

The poly herbal mixture of extracts was evaluated for parameters like bulk density, tapped density, Carr's index, Hausner's ratio and angle of repose by conventional method to determine the flow properties and compressibility of the prepared blend.

TABLE 3

| Parameter | Results | Inference |
|---|---|---|
| Bulk density (gm/cm3) | 0.53 | Passes |
| Tapped density (gm/cm3) | 0.0612 | Passes |
| Carr's index (%) | 13.39 | Good |
| Hausner's ratio | 1.15 | Good |
| Angle of repose | 20.7 | Good |

### C. Evaluation of the Polyherbal Tablet

TABLE 4

| Evaluation parameters | Result |
|---|---|
| Weight variation (mg) | 100 |
| Diameter (mm) | 12 |
| Thickness (mm) | 4 |
| Hardness (kg/cm2) | 6 |
| Friability (%) | 0.3-0.4 |
| Disintegration (sec) | 360 |

### D. Dissolution Studies for Tablet:

The in vitro drug dissolution studies were carried out using USP dissolution apparatus II under sink conditions. Phosphate buffer having pH 6.8 solution was used as a dissolution medium. The dissolution medium was prepared by dissolving 28.80 g of disodium hydrogen phosphate and 11.45 g of potassium dihydrogen phoshphate in sufficient amount of water to produce 1000 ml. The dissolution medium was continuously stirred at 100 rpm at 37±5° C. At different intervals of time 5 ml of samples were withdrawn and filtered and compensated by same volume of fresh medium. Samples were then measured in triplicates using UV spectrophotometer at 282 nm.

TABLE 5
Dissolution results of polyherbal tablet

| | ABSORBANCE AT |
|---|---|
| TIME (Mins) | 282 nm |
| 0 | 0.0191 |
| 5 | 0.4470 |
| 10 | 0.6297 |
| 15 | 0.6852 |
| 20 | 0.7865 |
| 25 | 0.9436 |
| 30 | 1.0252 |
| 45 | 1.1365 |
| 60 | 1.1364 |

### 2. Formulation of Polyherbal Capsules

a. Preparation of Powder Blend for Capsule:

The extracts of herbs were weighed and mixed thoroughly to ensure uniformity of the particles. The mixture was then passed through a #40 mesh sieve to achieve the desired particle size distribution. The powder was filled into capsule shells.

b. Evaluation of Capsules

The capsule size and weight were within the recommended pharmacopeial limits. The weight of individual capsules showed no significant difference as compared to the average capsule weight.

c. Dissolution Studies for Capsule:

The in vitro drug dissolution studies were carried out using USP dissolution apparatus I under sink conditions. Phosphate buffer having pH 6.8 solution was used as a dissolution medium. The dissolution mediums prepared by dissolving 28.80 g of disodium hydrogen phosphate and 11.45 g of potassium dihydrogen phosphate in sufficient amount of water to produce 1000 ml. The dissolution medium was continuously stirred at 100 rpm at 37±50° C. At different intervals of time, 5 ml of samples were withdrawn and filtered and compensated by same volume of fresh medium. Samples in triplicates were then measured using UV spectrophotometer at 282 nm.

TABLE 6
Dissolution results of polyherbal capsules

| Time | Absorbance |
|---|---|
| (Mins) | at 282 nm |
| 0 | −0.0063 ± 0.0004 |
| 5 | 0.2945 ± 0.0001 |
| 20 | 0.6070 ± 0.0002 |
| 25 | 0.9917 ± 0.0008 |
| 30 | 1.1666 ± 0.0005 |
| 45 | 1.2463 ± 0.0009 |
| 60 | 1.2053 ± 0.0006 |

### Example 3

The in-vitro anti-inflammatory effect of individual plant extracts of N. sativa, T. cordifolia, P. hybridus, and P. frutescens, as well as polyherbal mixture of the four extracts (composition of example 1) was studied. The ability of the individual extracts and the polyherbal mixture to inhibit protein denaturation was studied

Stock solutions of each of the individual extracts was made by dissolving 200 mg of each extract in 100 ml of distilled water. A stock solution of the polyherbal extract was prepared by dissolving 200 mg of the polyherbal mixture (described in Example 1 above) in 100 ml of distilled water. Standard solutions were solutions of ibuprofen, cetirizine and fexofenadine at a strength of 100 μg per ml each

5 gm of bovine serum albumin powder was dissolved in 100 ml of distilled water and was adjusted to pH 7 using 0.1M HCl, to obtain a 5% solution. One tablet of Phosphate Buffered Saline (PBS) was dissolved in 1000 ml of distilled water to obtain PBS solution of pH 7.2

2 mL of 5% bovine albumin was mixed with 0.12, 0.25, 0.3, 0.5, 1, 5, 10, 20, 40 and 80 micrograms of each of (1) the 4 individual extracts, (2) the polyherbal extract and (3) the standard solutions, and the final volume was made up with PBS to 5 ml. The mixture was then incubated in a water bath at 37° C. for 15 minutes, followed by heating at 70° C. for 5 minutes. The turbidity, if any, was made uniform by using a vortex. The solution was cooled and the turbidity was measured at 660 nm using a UV/VIS spectrometer (Optima, SP-3000, Tokyo, Japan). The PBS was used as the control. The percentage inhibition of protein denaturation was calculated by using the following formula:

$$\% inhibition of denaturation = [ ( Blank - Sample ) \times 100 ] / Blank$$

wherein
Blank=absorption of the control
Sample=absorption of the test sample of each individual extract and the polyherbal extract

The % inhibition calculated as above for each of the strengths for each of the extracts was plotted on a graph (% inhibition of protein coagulation versus concentration in micrograms) and the IC50 value for each extract was thus determined by calculating the slope of the graph.

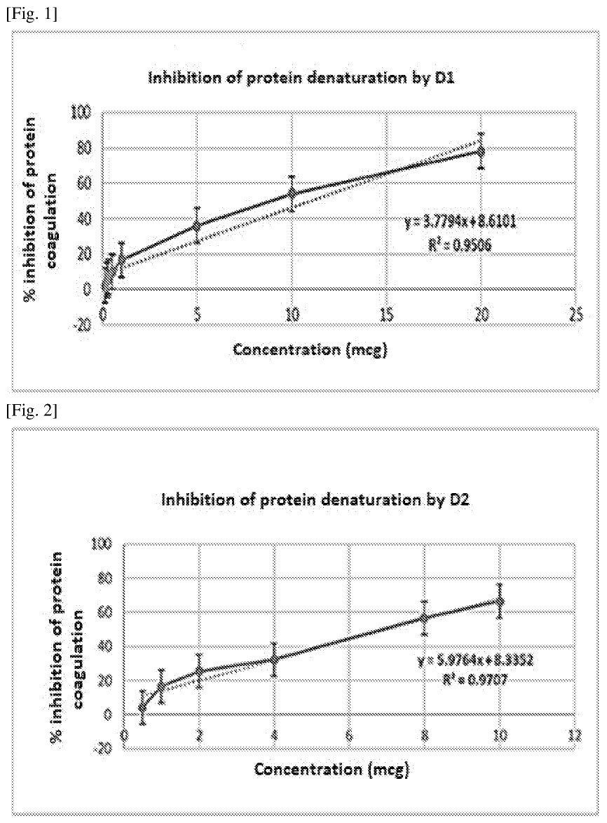

Inhibition of protein denaturation by Nigella sativa (Ranunculaceae) was found to be 2.31% to 84.34% at dose ranging from 0.12 μg to 80 μg, respectively. From the graph ([FIG. 1]) for extract of Nigella sativa, 50% inhibition of protein denaturation was found at concentration of 10.97 μg.

Inhibition of protein denaturation by Tinospora cordifolia (Menispermaceae) was found to be 4.4% to 66.55% at dose ranging from 0.5 μg to 10 μg, respectively. From the graph ([FIG. 2]), 50% inhibition of protein denaturation was found at 6.97 μg for Tinospora cordifolia (Menispermaceae).

Inhibition of protein denaturation by Petasites hybridus (Asteraceae) was found to be 2.31% to 78.12% at dose ranging from 0.12 μg to 20 μg, respectively. From the graph ([FIG. 3]), 50% inhibition of protein denaturation was found at 11.2 μg for Petasites hybridus (Asteraceae).

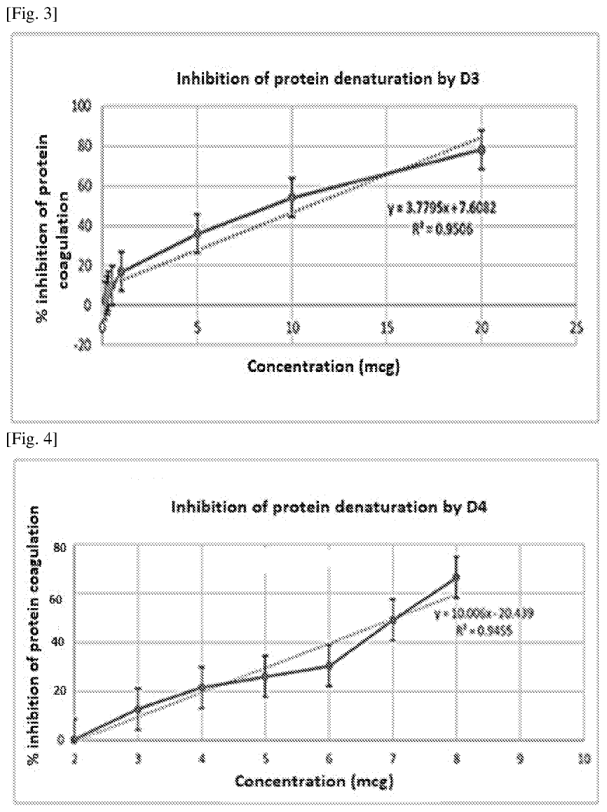

Inhibition of protein denaturation by Perilla frutescens (lamiaceae) was found to be 0.4% to 66.66% at dose ranging from 0.25 μg to 10 μg, respectively. From the graph ([FIG. 4]), 50% inhibition of protein denaturation was found at 7 μg for Perilla frutescens (lamiaceae).

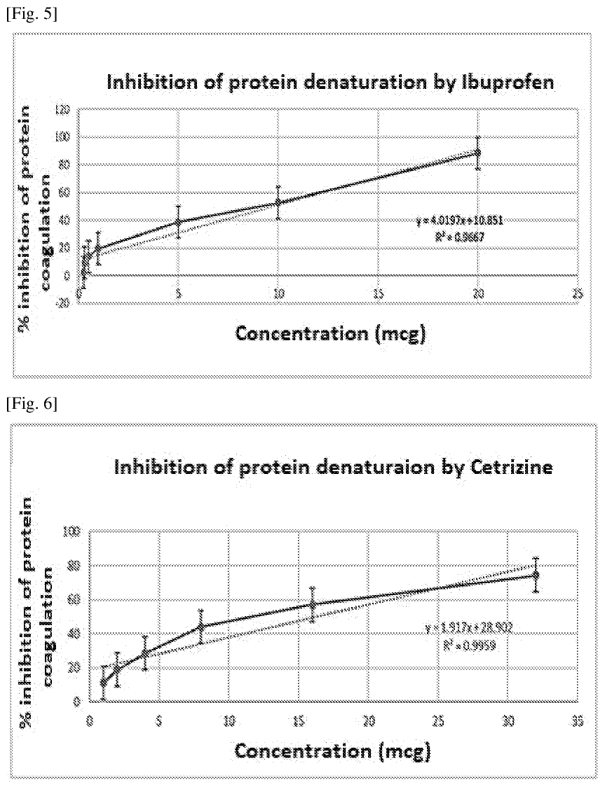

Inhibition of protein denaturation by ibuprofen was found to be 0.04% to 96.03% at dose ranging from 0.12 μg to 80 μg, respectively. From the graph ([FIG. 5]), 50% inhibition of protein denaturation was found at 9.73 μg for ibuprofen.

Inhibition of protein denaturation by cetirizine was found to be 11.23% to 74.28% at dose ranging from 1 μg to 32 μg, respectively. From the graph ([FIG. 6]), 50% inhibition of protein denaturation was found at 11 μg for cetirizine.

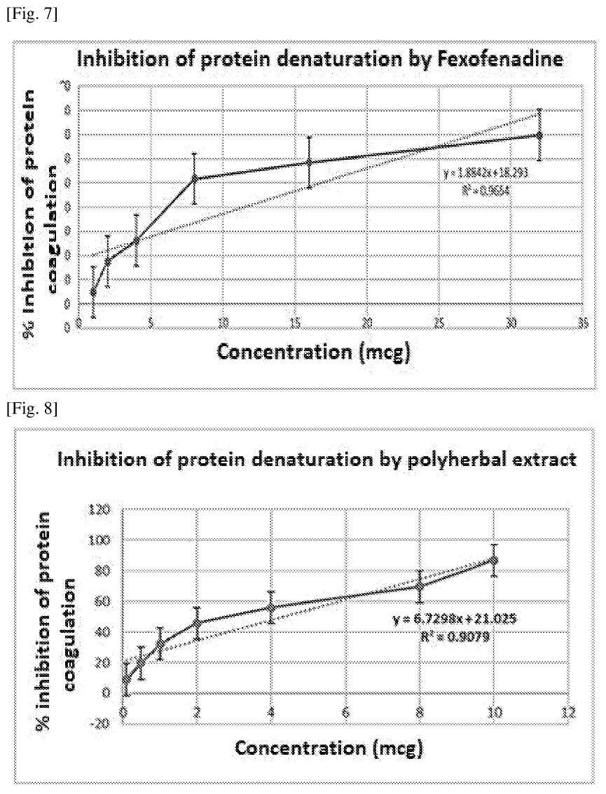

Inhibition of protein denaturation by fexofenadine was found to be 4.91% to 69.75% at dose ranging from 1 μg to 32 μg, respectively. From the graph ([FIG. 7]), 50% inhibition of protein denaturation was found at 16.83 μg for fexofenadine.

Inhibition of protein denaturation by polyherbal aqueous extract of example 1 was found to be 9.04% to 86.84% at dose ranging from 0.1 μg to 10 μg, respectively. From the graph ([FIG. 8]), 50% inhibition of protein denaturation was found at 4.3 μg for the polyherbal extract.

While all the extracts were found to possess a significant anti-inflammatory effect, the polyherbal extract exhibited anti-inflammatory effects greater than that of the effects produced by the individual extracts and were also better than the control standards. The Table 7 below provides details of the IC50 values obtained. It is amply clear that the polyherbal extract exhibits synergy as regards inhibition of protein denaturation, i.e., the inhibitory effect is much more than additive effect of the 4 extracts.

TABLE 7
IC50 values for individual extracts, polyherbal
mixture and control standards

| Compound tested | IC50 (μg) |
|---|---|
| N. sativa (Ranunculaceae) | 10.97 |
| T. cordifolia (Menispermaceae) | 6.97 |
| P. hybridus (Asteraceae) | 11.2 |
| P. frutescens (Lamiaceae) | 7 |
| Polyherbal extract (aqueous) | 4.3 |
| Ibuprofen | 9.73 |
| Cetirizine | 11 |
| Fexofenadine | 16.83 |

### Example 4

### Evaluation of Antiallergic/Anti-Inflammatory Activity of Herbal Extracts

The objective of this study is to evaluate antiallergic activity of polyherbal extract of example 1 by calculating eosinophils and leukocyte count from mouse model study against standard drugs, i.e. levocetirizine and fexofenadine.

Study was conducted to check the effect of extracts on milk-induced eosinophilia and leukocytosis in mice, by determining the count of eosinophils and leukocytes. For this model, 54 Swiss albino mice weighing in the range of 20-25 gm were used and divided in nine groups (6 mice/group). The eosinophils and leukocyte count was checked before and after treatment i.e. 24 hours after dosing with polyherbal formulation and standard drugs.

All the animals were maintained in the animal house under standard conditions: temperature (24±1° C.), relative humidity (45-55%), light (12 h) and dark (12 h) cycle, and were allowed free access to food and water ad libitum. After one-week acclimatization in the animal house, the animals were used for the experiments. Extracts were dosed as per standard protocol and animals were observed for signs and symptoms along with weekly body weight.

### Test Item Preparation:

Polyherbal extract containing Nigella sativa 88 mg/kg, Tinospora cordifolia 158 mg/kg, Petasites hybridus 26 mg/kg and Perilla frutescens 35 mg/kg was suspended in carboxy methyl cellulose. The suspension thus contained 307 mg/kg of the extract.

Similarly, standard drugs levocetirizine and fexofenadine were prepared as per requirement

### I] Animal Groups and Treatment Models of the Study Presented in Table 8

TABLE 8

| Sr. | Animal | Group | | Dose in | |
|---|---|---|---|---|---|
| No. | Group | Name | Treatment | animals | |
| 1 | Group 1 | Normal | CMC suspension | 10 | ml/kg p.o |
| | | Control (NC) | | | |
| 2 | Group 2 | Toxicant | Boiled and | 4 | ml/kg s.c |
| | | Group | cooled milk | | |

| | | | | |
|---|---|---|---|---|
| 3 | Group 3 | Polyherbal | Polyherbal | 307 mg/kg, |
| | | extract | extract + | p.o + |
| | | | boiled and | 4 ml/kg s.c |
| | | | cooled milk | |
| 4 | Group 4 | Levocetirizine | Levocetirizine + | 0.9 mg/kg, |
| | | | boiled and | p.o + |
| | | | cooled milk | 4 ml/kg s.c |
| 5 | Group 5 | Fexofenadine | Fexofenadine + | 21 mg/kg, |
| | | | boiled and | p.o + |
| | | | cooled milk | 4 ml/kg s.c |

The treatment of polyherbal extract and standard drug formulation were given orally 1 hour prior to injection of boiled and cooled milk. After 24 hours, blood samples were collected from retro orbital plexus, under ketamine xylazine anesthesia. Total eosinophil and differential leukocyte count per cu·mm was anlysed using hematology analyzer before and after treatment.

TABLE 9
Eosinophil and leukocyte count

| | Pre- | Post- | Pre- | Post- |
|---|---|---|---|---|
| | treatment | treatment | treatment | treatment |
| | Eosinophil (10³/μL) | | Total leucocyte count (10³/μL) | |
| Group | (mean ± SD) | | (mean ± SD) | |
| 1 | 0.17 ± 0.08 | 0.200 ± 0.063 | 7.25 ± 1.5 | 7.150 ± 0.938 |
| 2 | 0.22 ± 0.04 | 0.500 ± 0.11 | 7.20 ± 0.34 | 11.933 ± 0.350 |
| 3 | 0.23 ± 0.10 | 0.200 ± 0.089 | 6.88 ± 1.39 | 6.517 ± 1.534 |
| 4 | 0.18 ± 0.08 | 0.167 ± 0.082 | 6.95 ± 1.57 | 6.950 ± 1.089 |
| 5 | 0.17 ± 0.08 | 0.183 ± 0.075 | 7.87 ± 0.72 | 6.917 ± 0.436 |

Total eosinophil count: Eosinophil count was increased significantly in disease control animal group whereas polyherbal extract treated group was effective in suppressing increase of eosinophil count. The polyherbal extract was found to be more effective than the standard drugs, levocetirizine and fexofenadine.

Total leukocyte count: Leukocyte count was increased significantly in disease control animal group whereas polyherbal extract treated group was effective in suppressing the increase in leukocyte count. The polyherbal extract exhibited anti-inflammatory effects greater than that of the control standards.

1. An anti-inflammatory composition comprising a mixture of—
(i) about 20% to about 35% w/w of extract of Nigella sativa,
(ii) about 40% to about 60% w/w of extract of Tinospora cordifolia,
(iii) about 5% to about 15% w/w of extract of Petasites hybridus,
(iv) about 5% to about 20% w/w of extract of Perilla frutescens.

2. The anti-inflammatory composition as claimed in claim 1, wherein the composition is in the form of a dosage form selected from the group comprising capsules, tablets, granules, powders filled in sachet, oral liquid solutions, oral liquid suspensions and dry suspensions for reconstitution.

3. The anti-inflammatory composition as claimed in claim 2, wherein the composition is a tablet.

4. The anti-inflammatory composition as claimed in claim 2, wherein the composition is a capsule.

5. The anti-inflammatory composition as claimed in claim 3, wherein the tablet further comprises excipients selected from one or more of binders, diluents, disintegrants, lubricants, flow-enhancing agents, solubility enhancing agents, permeability enhancing agents, sweeteners, flavorants and coating agents.

6. The anti-inflammatory composition as claimed in claim 4, wherein the capsule further comprises excipients selected from one or more of binders, diluents, disintegrants, lubricants, flow-enhancing agents, solubility enhancing agents, permeability enhancing agents, sweeteners and flavorants.

7. The anti-inflammatory composition as claimed in claim 1, wherein the composition contains from about 0.12 μg to about 80 μg of Nigella sativa.

8. The anti-inflammatory composition as claimed in claim 1, wherein the composition contains from about 0.5 μg to about 10 μg of Tinospora cordifolia.

9. The anti-inflammatory composition as claimed in claim 1, wherein the composition contains from about 0.12 μg to about 20 μg of Petasites hybridus.

10. The anti-inflammatory composition as claimed in claim 1, wherein the composition contains from about 0.25 μg to about 10 μg of Perilla frutescens.

11. The anti-inflammatory composition as claimed in claim 1, wherein the composition is used in the treatment and management of allergies.

12. The anti-inflammatory composition as claimed in claim 1, wherein the composition contains about 0.12 μg to about 80 μg of Nigella sativa, about 0.5 μg to about 10 μg of Tinospora cordifolia, about 0.12 μg to about 20 μg of Petasites hybridus and about 0.25 μg to about 10 μg of Perilla frutescens.

13. The anti-inflammatory composition as claimed in claim 12, wherein the composition possesses IC50 value of about 4.3 μg.
